(19) United States
(12) Patent Application Publication
(10) Pub. No.: US 2018/0000095 A1
(43) Pub. Date: Jan. 4, 2018

(54) MICROBICIDAL OIL-IN-WATER DISPERSION

(71) Applicant: REPOLAR PHARMACEUTICALS OY, Espoo (FI)

(72) Inventor: Pentti SIPPONEN, Espoo (FI)

(21) Appl. No.: 15/544,230

(22) PCT Filed: Jan. 19, 2016
(86) PCT No.: PCT/FI2016/050022

§ 371 Date: Jul. 17, 2017

(30) Foreign Application Priority Data
Jan. 20, 2015 (FI) 20155046

(51) Int. Cl. A01N 65/06; A01N 25/04; A61K 9/107; A61K 36/13

(52) U.S. Cl. CPC A01N 65/06; A61K 36/13; A01N 25/04; A61K 9/107

(57) ABSTRACT

The invention relates to an oil-in-water dispersion comprising coniferous resin acids, its preparation and use as an antimicrobial and anti-inflammatory agent in medical and non-medical products. The invention also relates to a pharmaceutical product comprising the oil-in-water dispersion.

### FIELD OF THE INVENTION

The present invention relates to an aqueous antimicrobial oil-in-water dispersion (emulsion), comprising coniferous resin acids, a method for its preparation and its use as an antimicrobial and anti-inflammatory agent in medical and non-medical products. The invention further relates to a pharmaceutical formulation comprising said dispersion.

### BACKGROUND OF THE INVENTION

Coniferous resin, typically of spruce and pine origin, has been widely used in traditional medicine for healing of skin wounds and skin infections. In this application, the resin is typically cooked (mixed) into animal fat. Resins or rosins mixed with fat (animal fat, butter etc.) are strongly antimicrobial and microbicidal against a large range of bacteria, fungi, molds, and even against viruses, and are particularly used in treatment of microbial wound infections.

Terms resin and rosin are disorderly used in the literature. Resin is used, for example, for a specific type of plastics, various synthesized polymers, and also as a term indicating various exudates or extracts of the vegetable kingdom in general. In addition to the term ‘resin’, such terms as ‘rosin gum’ or ‘colophonium’ are widely used as synonyms to the resin.

The antimicrobial characteristics, such as antifungal and antibacterial properties, and even the antiviral characteristics of coniferous resin, are described in the literature. It has been shown that particularly the resin acids in resin and rosin have antimicrobial potentials. For example, coniferous resin has broad-spectrum antimicrobial characteristics against pathogenic gram-positive and gram-negative bacteria.

Coniferous resins and rosins are extremely poorly soluble in water but dissolve into alcohol or organic solvents to some extent. If the resin/rosin, or resin acids, are mixed with pure water, the resultant water solution is not antimicrobial. If resin/rosin compounds dissolved in an alcoholic solution are mixed with water, they are precipitated, and the resultant water fraction is not antimicrobial.

There are fat-based resin salves available in the market, which are shown to be effective in the treatment of skin wounds and fungal nail infections. These salves are based on a mixture of the resin or rosin with various fats.

WO 2011/042613 A2 describes an antimicrobial composition comprising coniferous resin, rosin, resin acids and/or their derivates in alcohol.

WO 2013/060936 A2 discloses an aqueous antimicrobial composition comprising coniferous resin acids and a dispersing agent. Resin or rosin is first dispersed with a dispersing agent, such as glycerol, after which an aqueous medium (e.g., physiological saline) is added to provide an antimicrobial aqueous dispersion that contains water miscible components from resin or rosin. However, since glycerol is hydrophilic, glycerol-containing resin/rosin compositions may not be suitable in all applications, especially in medical applications. The use of the glycerol-containing compositions is thus restricted.

Extensive use of coniferous resins or rosins as antimicrobial agents particularly in the treatment of humans and animals in a wide variety is not possible in large entity until aqueous, water miscible solutions comprising resin or rosin can be provided. Thus, there is a need for aqueous and water miscible compositions which are antimicrobial and anti-inflammatory, comprising water “soluble” components from coniferous resin or rosin.

### BRIEF DESCRIPTION OF THE INVENTION

We have surprisingly found that a stable, aqueous composition of coniferous resin or rosin having antimicrobial and anti-inflammatory properties can be provided by first dissolving coniferous resin or rosin into oil and then adding an aqueous medium to the oil. A stable oil-in-water dispersion comprising coniferous resin acids is obtained which can be further diluted with an aqueous diluent, solvent or excipient, such a saline solution, a sugar solution, aqueous infusion and injections solutions, without any deposit formation.

In an aspect, the present invention provides an oil-in-water dispersion comprising coniferous resin acids, wherein the concentration of the coniferous resin acids in the oil-in-water dispersion is in the range of about 1 ppm to about 5000 ppm.

In another aspect, the present invention provides a method for producing an oil-in-water dispersion comprising coniferous resin acids, comprising the steps of:
providing coniferous resin acids,
dissolving the coniferous resin acids into oil to provide a mixture,
adding an aqueous medium to the mixture to provide an oil-in-water dispersion layer and a water-in-oil dispersion layer,
separating and recovering the oil-in-water dispersion layer.

In a further aspect, the present invention provides use of the oil-in-water dispersion of the invention, comprising coniferous resin acids, or that prepared by the method of invention as an antimicrobial agent in non-medical applications.

In a still further aspect, the present invention provides the oil-in-water dispersion of the invention, comprising coniferous resin acids, or that prepared by the method of invention for use as an antimicrobial agent or an anti-inflammatory agent for treating or preventing infections and/or inflammations in humans and animals.

In a still further aspect, the present invention provides a pharmaceutical formulation comprising oil-in-water dispersion of the invention, comprising coniferous resin acids, or that prepared by the method of the invention.

Also a method of treating inflammations and/or infections in humans and animals or of preventing disorders therein, said method comprising administering an oil-in-water dispersion of the invention, comprising coniferous resin acids, or that prepared by the method of invention to said subject in therapeutic or preventive amounts, is described.

### BRIEF DESCRIPTION OF THE DRAWINGS

FIGS. 1-4 show the antimicrobial effect of oil-in-water emulsions of the invention.

### DETAILED DESCRIPTION OF THE INVENTION

In an aspect, the present invention provides an oil-in-water dispersion comprising coniferous resin acids, wherein the concentration of the coniferous resin acids in the oil-in-water dispersion is in the range of about 1 ppm to about 5000 ppm.

In another aspect, the present invention provides a method for producing an oil-in-water dispersion comprising coniferous resin acids, comprising the steps of:
providing coniferous resin acids,
dissolving the coniferous resin acids into oil to provide a mixture,
adding an aqueous medium to the mixture to provide an oil-in-water dispersion layer and a water-in-oil dispersion layer,
separating and recovering the oil-in-water dispersion layer.

The oil-in-water dispersion of the invention and that prepared by the method of the invention are stable, evenly dispersible to and dilutable with an aqueous medium.

The oil-in-water dispersion of the invention and that prepared by the method of the invention have antimicrobial and anti-inflammatory properties. It is to be noted that the anti-inflammatory effect is other than the antimicrobial effect, and indicates the sterile reaction of biological tissues and cells.

As stated above, terms resin and rosin are disorderly used in the literature. In the present invention the term “coniferous resin” indicates native callus resin, which is solid or highly viscous material, exuded from the callus tissue onto the wounded surface of the tree trunks of conifers. Native callus resin on the tree trunks is typically accumulated as multicolored clods. Coniferous resin is a mixture of various chemical compounds, including coumaric acids, resin acids, lignans, fatty acids and volatile terpenic compounds.

The term “rosin” means a resin acid fraction obtained from distillation of tall oil obtained as a by-product from kraft pulping of coniferous trees. The rosin is enriched with various resin acids but is substantially free of lignans and coumaric acids present in native callus resin. However, the rosin fraction may contain minor amounts of fatty acids (1-3% w/w). Commercial rosin products are available in the market.

The coniferous resin acids can be provided as coniferous resin, rosin, or as a mixture thereof in the dispersion and in the method of the invention.

Coniferous resin acids used in the present invention can be derived from various sources. In an embodiment, coniferous resin acids are provided as native coniferous resin in solid or highly viscous form collected from coniferous tree trunks. The coniferous resin can be purified so as to provide a resin fraction enriched with resin acids which is substantially free of impurities, such as bark and grains of sand, and of lignans and fatty acids. In another embodiment, the coniferous resin acids are provided as rosin. In a further embodiment, coniferous resin acids are provided as a fraction of specific resin acids isolated from a composition comprising a wide variety of coniferous resin acids. Coniferous resin acids can also be provided as a mixture from various sources.

The coniferous resin acids can be provided as a form ranging from solution to powder or granulates, having a varied solid matter content.

Coniferous resin acids are organic acids present in resin and rosin. These acids include water soluble resins acids, for example, dehydroabietic acid, 7β-hydroxydehydroabietic acid, 7α-hydroxydehydroabietic acid, 15-hydroxydehydroabietic acid, 7β,15-dihydroxydehydroabietic acid, 7α,15-hydroxydehydroabietic acid, 18-hydroxydehydroabietic acid, further hydroxylated derivates of dehydroabietic acid and a mixture thereof.

The coniferous resin acids are dissolved in oil. Any oil which is not harmful to human and animal health can be used in the present invention. Suitable oil includes, but is not limited to, mineral oil, organic oil, such as plant oil, vegetable oil, animal fat, for example, paraffin, petrolatum, palm oil, flaxseed oil, rape-seed oil, sunflower oil, avocado oil, sesame oil, nut oil and olive oil. A mixture of the oils can also be used. At least a portion of the resin acids is dissolved in oil.

The coniferous resin or rosin is typically introduced into oil in a concentration of about 1% to about 50% (w/w). In an embodiment, the concentration of resin or rosin is about 5% to about 30%. In a further embodiment, the concentration is about 10% to about 20% (w/w).

In an embodiment, prior to dissolution of the coniferous resin or rosin into oil, resin or rosin is first dissolved in a volatile organic solvent, such as alcohol or acetone, to provide a purified resin fraction. Any suitable alcohol can be used including, but not limited to, ethanol. This purification is particularly suitable when native callus resin is employed as a starting material. Any solid matter insoluble in the organic solvent can be filtered off. The volatile solvent is left to evaporate to provide viscous purified resin enriched with resin acids. Evaporation can be enhanced by heating, if desired. The solid matter of the purified resin fraction is typically at least about 60% (w/w).

The aqueous medium added to the oil in which coniferous resin acids are dissolved can be any aqueous solution. In an embodiment, the aqueous medium is a physiological saline solution. The physiological saline solution is preferred, when the antimicrobial composition is used in applications in fields of human and veterinary medicines. In another embodiment, the aqueous medium is water.

The aqueous medium is added to the mixture of oil and coniferous resin acids in a volume ratio of about 1:50 to about 50:1. In this step, water soluble resin acids are passed from oil into an aqueous medium. In an embodiment, the aqueous medium is added in a volume ratio of up to about 1:15. In another embodiment, the volume ratio is about 1:1 (vol/vol).

By adding an aqueous medium to oil, two separate layers, i.e. an upper oil-in-water dispersion layer (aqueous layer) and a lower water-in-oil dispersion layer (oil layer), are obtained. The oil-in-water dispersion means an emulsion in which oil is dispersed in a continuous phase of an aqueous medium. The oil-in water dispersion is mainly composed of water, having a minor portion of small oil droplets dispersed in water. The water-in-oil dispersion means an emulsion in which water is dispersed in a continuous phase of oil. The water-in-oil dispersion is mainly composed of oil, having a minor portion of small water droplets dispersed in oil. Both emulsions beneficially contain coniferous resin acids which are soluble in oil, on one hand, and in an aqueous medium, on the other hand.

In an embodiment, after addition of an aqueous medium to oil, the whole oil-aqueous medium composition is shaken or mixed in order to enhance the dispersibility of coniferous resin or rosin in the oil-in-water dispersion. Also, heat treatment can be applied for these purposes.

After addition of an aqueous medium to oil, the whole oil-aqueous medium composition, optionally shaken, mixed and/or heat-treated, is left to stabilize typically for 1-12 hours to provide the two separate dispersion layers.

Any suitable emulsifying agent can be added to enhance emulsification of coniferous resin or rosin in the oil-in-water dispersion (emulsion). An example of suitable emulsifying agents is polysorbates.

Any solid matter insoluble either in the oil or in the aqueous medium and precipitated between the upper aqueous layer and the lower oil layer can be filtered off.

Finally, an evenly distributed and stable colloidal oil-in-water dispersion (emulsion) is obtained. This dispersion can be further diluted with an aqueous solution, such as with saline, sugar solution, aqueous infusion and injections solution, without formation of any precipitations or deposits. The antimicrobial effect of the dispersion can be adjusted by diluting the dispersion with an aqueous medium. The dispersion can be diluted, for example, in a volume ratio of up to about 1:6. In an embodiment, the dispersion is diluted in a volume ratio of 1:3. In another embodiment, the dispersion is diluted in a volume ratio of 1:4.

The concentration of the coniferous resin acids in the oil-in-water dispersion of the invention and that prepared by the method of the invention is in the range of about 1 ppm to about 5000 ppm. In another embodiment, the concentration is about 1 ppm to about 2000 ppm. In still another embodiment, the concentration is about 1 ppm to about 500 ppm. In a further embodiment, the concentration is about 10 ppm to about 100 ppm.

The oil-in-water dispersion of the invention and that prepared by the method have antimicrobial and anti-inflammatory properties. Also, the water-in-oil dispersion obtained in the method of the invention shows antimicrobial and anti-inflammatory properties.

The dispersion of the invention is free of highly volatile organic compounds, such as ethanol.

In a further aspect, the present invention provides a use of an oil-in-water dispersion of the invention, comprising coniferous resin acids, or that prepared by the method of the invention as an antimicrobial agent or a preservative in non-medical application. The dispersion can be used as in antimicrobial, protective, preventive anti-inflammatory (antioxidative) or protective purposes in various industrial materials and products, biocides, washing agents, cosmetics, hygiene products, paints, coatings, plastics, and in foodstuffs.

The oil-in-water dispersion of the invention, comprising coniferous resin acids, or that prepared by the method of the invention is suitable for use as a medicine as such or as an additive ingredient in pharmaceutical formulations intended to be applied in treatment of inflammation (anti-inflammatory property), including sterile inflammation, and/or infection (antimicrobial property) in mammals. Thus, in an aspect, the invention provides an oil-in-water dispersion of the invention or that prepared by a method of the invention for use as an antimicrobial agent or an anti-inflammatory agent for treating or preventing infections and/or inflammations in humans and animals.

In an embodiment, the oil-in-water dispersion of the invention, or that prepared by the method of the invention is used for treating or preventing inflammation and/or infection caused by Helicobacter pylori.

In a still further aspect, the present invention provides a pharmaceutical formulation comprising oil-in-water dispersion of the invention, comprising coniferous resin acids, or that prepared by the method of the invention. The pharmaceutical formulation can be provided as a tablet, capsule, pill, solution, infusion or injection liquid, clysma (enema), washing agent, shampoo, drop, stick, spray, salve, cream, gel, ointment, lotion, lacquer, enema, vagitorium or suppositorium. The pharmaceutical formulation may also comprise conventional excipients and/or functional additives typically used in a specific formulation. In an embodiment, pharmaceutical formulations are aqueous pharmaceutical formulations. In an embodiment, the pharmaceutical formulation is eye drops.

The pharmaceutical formulation may be administered orally, intravenously, intra-arterially, subcutaneously, intramuscularly, topically or as an injection or infusion.

A wide range of various bacteria can cause stomach infections (chronic gastritis) that can be treated with the antimicrobial dispersion of the invention. The dispersion is microbicidal in a wide range of micro-organisms, including fungi, molds and even viruses. Helicobacter pylori (Hp) is one example of clinically important microbes sensitive to resin containing compositions. Hp is the cause of chronic gastritis and chronic gastritis in stomach mucosa is the basic reason for the most important gastric disorders, such as stomach cancer or peptic ulcer, atrophic gastritis and hypochlorhydric stomach. It is widely known that the treatment against Helicobacter pylori is problematic which may require several re-attempts and many antibiotics. Helicobacter pylori infections are typically treated also with colloidal bismuth compositions that topically (intragastrically) kill bacteria that grow and multiplicate on the stomach mucosa surface.

The oil-in-water dispersion of the invention or that prepared by the method of the invention is applied in medicines and pharmaceutical formulations or products, for example, as follows:
as an antimicrobial or anti-inflammatory therapeutic agent (tablets, capsules, infusion and injections liquids, salves, creams, emulsions, drops, gels, suppositorium, etc.) with application of the dispersion as such or by replacing the aqueous components of the medicines by the coniferous resin oil-in-water dispersion
as an antimicrobial fluid to be injected into tissues, tissue cavities, joint, or abscesses as described above by applying the dispersion as such or by replacing the aqueous components of the medicines by the coniferous resin oil-in-water dispersion
as an antimicrobial solution for washing of tissue cavities (oral cavity, maxillary sinus, ear canal, abdominal cavity, pleural cavity, urinary bladder and urinary tract or vagina, fistulae, fissures, or ducts, etc.) by applying the dispersion as such or by replacing the aqueous components of the medicines by the coniferous resin oil-in-water dispersion
as an antimicrobial composition in treatment of skin ulcers, sores, burns, or infections by applying the dispersion as such or by replacing the aqueous components of the medicines by the coniferous resin oil-in-water dispersion
as an antimicrobial additive in bandages, dressings, tissue towels or products used in treatment of skin ulcers, sores, burns or infections by impregnating the coniferous resin oil-in-water dispersion into these dressings
as an antimicrobial composition in topical medicines for fungal nail or skin infections by applying the coniferous resin oil-in-water dispersion as the medicine by itself or by replacing the aqueous components of the medicines (salves, creams, drops, solutions, etc) with the coniferous resin oil-in-water dispersion
as eye drop, eye salve or eye washing solution by applying the dispersion as such or by replacing the aqueous components of the medicines by the coniferous resin oil-in-water dispersion
as mouthwash by adopting the dispersion as such or by replacing the aqueous components of the mouthwash by the coniferous resin oil-in-water dispersion
as vagitorium, suppositorium, gel, cream or solution in treatment of gynecological infections or diseases (bacterial and fungal infections) adopting the dispersion as such or by replacing the aqueous components of the products by the coniferous resin oil-in-water dispersion
as an additive in water-dilutable creams, solutions, lotions, gels, sprays, soaps or medicaments used in treatment, desinfection or washing of skin, skin wounds, skin lesions or infections
as an antimicrobial additive in cosmetic or hygiene products, tissue papers, soaps or shampoos
as an antimicrobial additive for preservation of medicines
as an antimicrobial agent in treatment of outer surfaces of a mammal, such as skin, hair, feet hoofs and udders by adopting the dispersion as such or by replacing the aqueous components of the medicine by the coniferous resin oil-in-water dispersion
as an antimicrobial additive in washing and protection of the outer surfaces of mammals, such as skin, hair, feet hoofs and udders by adopting the dispersion as such or by replacing the aqueous components of the medicine by the coniferous resin oil-in-water dispersion.

In order to avoid usage of antibiotics and corticosteroids, an oil-in-water emulsion of the invention or that prepared by the method of the invention can be administered to humans or animals for treating inflammations and/or infections or preventing disorders therein in therapeutically effective or preventive amounts.

The content of the coniferous resin acids in such products is typically in the range of about 1 to about 30 ppm.

In non-medical purposes, the oil-in-water dispersion of the invention or that prepared by the method of the invention can be applied as follows:
as an antimicrobial additive or preservative in paints, lacquers, varnish, wax, or various other cover materials
as a microbicidal agent for cleaning of medical instruments such as catheter, prosthesis, implants, etc.,
as an antimicrobial additive or preservative in cleaning compositions (washing solutions), disinfectants and toiletry, such as shampoo, soap, deodorant, mouthwash or toothpaste
as an antimicrobial additive or preservative in cosmetics,
as an antimicrobial composition for purification of solutions, such as drinking water, water reservoirs
as an antimicrobial foodstuff preservative
as biocide or pesticide.

The dispersion of the invention can be applied to the product by coating, spraying, impregnating, brushing, spreading, wetting, etc. Food products (foodstuff) can be protected by spreading the dispersion on surface of the foodstuff or by including the composition into a washing solution. Since the dispersion is water miscible, the dispersion can be washed out by water from food product before use.

The dispersion of the invention can be applied as a biocide, for example, in formulation of a washing solution or a spray. Fungus-like oomycetes Phytopthora infestans (cause of the potato blight) or fungus Rhizoctonia solani (causing collar rot, root rot in plants) are sensitive to the aqueous resin dispersions, against which the composition can be applied as a spray or washing solution.

The following examples are given for further illustration of the invention without limiting the invention thereto.

### Example 1

Native callus resin was mixed with ethanol and let to stand for 3 to 4 weeks. Non-dissolved matter was filtered off. A 60% resin solution in ethanol was obtained. 100 ml of the ethanol solution (TS 60%) was mixed with 1000 ml rapeseed oil. Ethanol was left to evaporate, and 1000 ml saline was added. The oil-saline solution was left to stabilize for 1-12 hours whereby an oil-in-water dispersion layer (upper layer) and an water-in-oil dispersion layer (lower layer) were formed. The oil-in-water dispersion (emulsion) was filtered off for the applications defined in the invention.

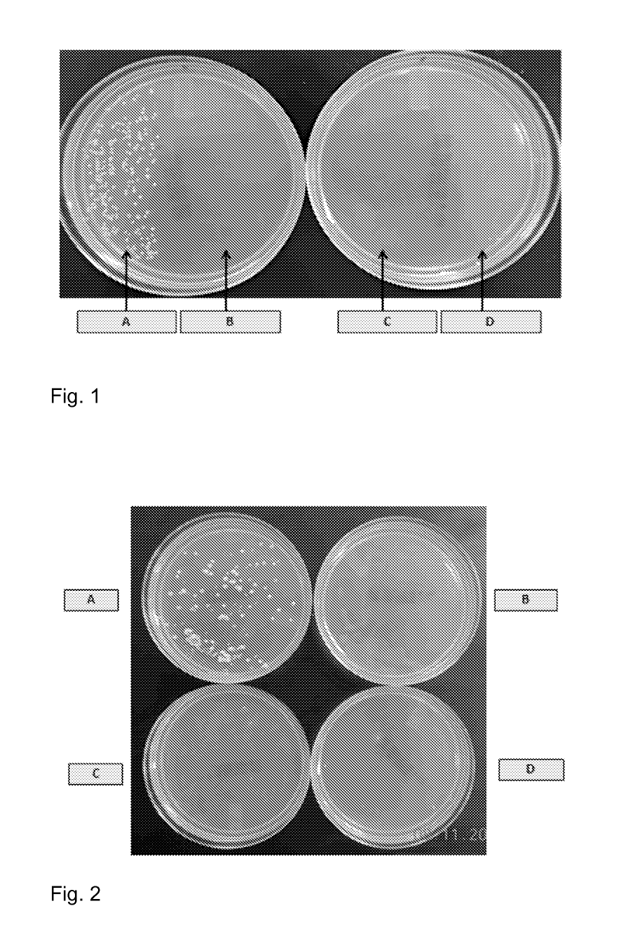

The antimicrobial effect of the oil-in-water dispersion obtained is shown in FIG. 1. The oil-in-water dispersion obtained above can be used in the following application as follows, however without limiting the applications thereto:

The oil-in-water dispersion is diluted to 20% (1:4) with physiological saline. Eye drops, wherein the aqueous part of the drop formulation is said 20% dilution are given to patients having dry eye syndrome.

The oil-in-water dispersion can be impregnated into washing bandages and applied as antimicrobial washing towels for humans and animals. The dispersion can also be used as a solution or as an ingredient in a gel or shampoo in treatment of skin psoriasis.

The oil-in-water dispersion can be used to replace an aqueous part (water) of the ingredients in pharmaceutical and cosmetic products in order to improve the preservation or microbicidal activity of the products. Addition of rapeseed oil-saline emulsion comprising coniferous resin makes the dispersion microbicidal and antibacterial in dilutions of 25% (vol/vol) or more (FIG. 4) and can be applied as an antibacterial and antifungal ingredient in vagitoriums.

The oil-in-water dispersion was diluted with a physiological saline solution in volume ratios of 1:1 and 1:3. These solutions can be used as an antimicrobial injection agent for inflamed joint, such as knee joint, or as an antimicrobial injection in body cavities or abscesses. The solution contains pharmaceutically active anti-inflammatory resin/rosin ingredients that are equally potential as dexamethasone in the anti-inflammatory property. The antimicrobial effect of the oil-in-water dispersion, and the two diluted solutions are shown in FIG. 4.

### Example 2

An oil-in-water dispersion was prepared as described in Example 1 except that instead of coniferous resin, rosin FOR90 fraction of tall oil from Forchem Oy was used.

The oil-in-water dispersion was diluted with a physiological saline solution in volume ratios 1:3 and 1.6. The antimicrobial effect of the oil-in-water dispersion, and the two diluted solutions are shown in FIG. 2.

### Example 3

An oil-in-water dispersion was prepared as described in Example 1 except that instead of rapeseed oil, olive oil was used. The antimicrobial effect of the oil-in-water dispersion obtained is shown in FIG. 1.

### Example 4

An oil-in-water dispersion was prepared as described in Example 1 except that instead of coniferous resin and rapeseed oil, rosin and olive oil, respectively, were used. The rosin was FOR90 fraction of tall oil from Forchem Oy. The antimicrobial effect of the oil-in-water dispersion obtained is shown in FIG. 1. The dispersion can be used as such, or as a diluted ingredient, in biocide or pesticide.

### Example 5

An oil-in-water dispersion was prepared as described in Example 1 except that flaxseed oil was used as a dispersing agent instead of rapeseed oil. The oil-in-water emulsion was used as such, or as a saline dilution (1/1 (vol/vol)) in skin diseases as an antifungal and anti-inflammatory lotion and liniment (psoriasis, dandruff, etc).

### Example 6

An oil-in-water dispersion was prepared as described in Example 5 except that rosin (FOR90) was used as a dispersing agent instead of coniferous resin.

### Example 7

An oil-in-water dispersion was prepared as described in Example 1 and was used as a spray or washing agent in treatment or prevention of the potato blight (Phytophthora infestans) against which the aqueous resin dispersion is microbicidal.

### Example 8

The content of various compounds included in the the oil-in-water dispersions of Example 1 (native callus resin) and Example 2 (rosin) were analyzed by short column gas chromatography (GC) and gas chromatography-mass spectrometry (GC-MS). The compounds were identified based on retention time and GC-MS. The quantity was determined by GC with betulin as a standard compound.

The results given in Table 1 below show that the resin acids are predominantly in an oxidized form in the oil-in-water dispersions.

TABLE 1

| Compound | Example 1 | Example 2 |
|---|---|---|
| p-coumaric | 232 ppm | 0 ppm |
| resin acids | traces | 0 ppm |
| oxidized resin acids | 116 ppm | 1435 ppm |
| Lignans | 215 ppm | 0 ppm |

Identified lignans were pinoresinol, lariciresinol, isolariciresinol and secoisolariciresinol.

The following oxidized resin acids were identified: 7β-hydroxydehydroabietic acid, 7α-hydroxydehydroabietic acid, 15-hydroxydehydroabietic acid, 7β,15-dihydroxydehydroabietic acid and 7α,15-dihydroxydehydroabietic acid.

### Microbiological Tests

The antimicrobial effect of the oil-in-water dispersions (emulsions) of the invention are illustrated in FIGS. 1-4. The antimicrobial activity of the dispersions against mouth microbes was measured by applying the Ph.Eur.5.1.3 challenge test (EN 13697). In the test, the test microbes were added into a test solution, i.e. the oil-in-water dispersion of the invention. Samples of the test solution were re-cultured in specific culture media (e.g., Mueller Hinton agar culture plate) to demonstrate whether the test solution under testing killed, or did not kill, the microbes during the incubation (challenge) period. This killing activity (microbicidal activity) was monitored by calculating the reduction of re-growth of microbes in the test samples after 24 hours. No growth of microbes (all microbes are killed in 24 hours) in the re-culture was considered to indicate the microbicidal activity of the oil-in-water dispersion of the invention.

The tested emulsions in FIGS. 1-4 were as follows:

In FIG. 1,

A: Reference emulsion. The reference emulsion was prepared as follows:

A physiological saline solution was mixed with olive oil in volume ratio of 1:1. The oil-saline solution was left to stabilize for 1-12 hours whereby a water-in-oil dispersion layer (upper layer) and an oil-in-water dispersion layer (lower layer) were formed. The oil-in-water dispersion (emulsion) was filtered off to provide the reference emulsion.

B: Oil-in-water dispersion of Example 4.
C: Oil-in-water dispersion of Example 3.
D: Oil-in-water dispersion of Example 1.

In FIG. 2,

### A: Rapeseed oil.

B: Oil-in-water dispersion of Example 2.
C: Diluted solution of Example 2 (1:3).
D: Diluted solution of Example 2 (1:6).

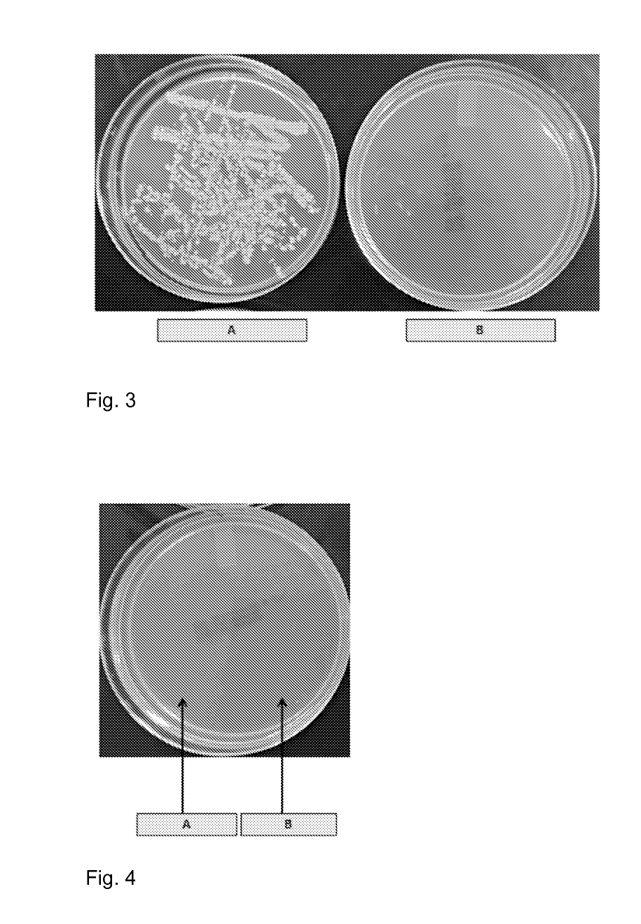

In FIG. 3,

### A: Flaxseed oil.

B: Oil-in-water dispersion of Example 6.

In FIG. 4,

A: Diluted solution of Example 1 (1:1).
B: Diluted solution of Example 1 (1:3).

1. An oil-in-water dispersion comprising coniferous resin acids, wherein the concentration of the coniferous resin acids in the oil-in-water dispersion is in the range of about 1 ppm to about 5000 ppm, specifically about 1 ppm to about 2000 ppm, more specifically about 1 ppm to about 500 ppm, still more specifically about 10 ppm to about 100 ppm.

2. The dispersion of claim 1, wherein the coniferous resin acids are provided as coniferous resin or rosin.

3. The dispersion of claim 1, wherein the coniferous resin acids include dehydroabietic acid, 7β-hydroxydehydroabietic acid, 7α-hydroxydehydroabietic acid, 15-hydroxydehydroabietic acid, 7β,15-dihydroxydehydroabietic acid, 7α,15-dihydroxydehydroabietic acid, 18-hydroxydehydroabietic acid, further hydroxylated derivates of dehydroabietic acid or is a mixture thereof.

4. The dispersion of claim 1, wherein the oil is mineral oil, organic oil, such as plant oil, vegetable oil, animal fat, paraffin, petrolatum, palm oil, flaxseed oil, rape-seed oil, sunflower oil, avocado oil, sesame oil, nut oil or olive oil, or a mixture of these oils.

5. A method for producing an oil-in-water dispersion comprising coniferous resin acids, comprising the steps of:
providing coniferous resin acids,
dissolving the coniferous resin acids into oil to provide a mixture,
adding an aqueous medium to the mixture to provide an oil-in-water dispersion layer and a water-in-oil dispersion layer,
separating and recovering the oil-in-water dispersion layer.

6. The method of claim 5, wherein the coniferous resin acids are provided as coniferous resin or rosin.

7. The method of claim 5, wherein the coniferous resin acids include dehydroabietic acid, 7β-hydroxydehydroabietic acid, 7α-hydroxydehydroabietic acid, 15-hydroxydehydroabietic acid, 7β,15-dihydroxydehydroabietic acid, 7α,15-dihydroxydehydroabietic acid, 18-hydroxydehydroabietic acid, further hydroxylated derivates of dehydroabietic acid or is a mixture thereof.

8. The method of claim 5, wherein the oil is mineral oil, organic oil, such as plant oil, vegetable oil, animal fat, paraffin, petrolatum, palm oil, flaxseed oil, rape-seed oil, sunflower oil, avocado oil, sesame oil, nut oil or olive oil, or a mixture of these oils.

9. The method of claim 5, wherein coniferous resin or rosin is introduced to the oil in a concentration of about 1% to about 50% (w/w), specifically about 5% to about 30%, more specifically about 10% to about 20%.

10. The method of claim 5, wherein the resin acids are first dissolved in a volatile organic solvent, such as alcohol, to provide a purified resin fraction which is then dissolved in the oil.

11. The method of claim 10, wherein a solid matter of the purified resin fraction is at least about 60% (w/w).

12. The method of claim 10, wherein any insoluble solid matter formed in the dissolution is filtered off.

13. The method of claim 5, wherein the aqueous medium is any aqueous solution, such as a physiological saline solution or water.

14. The method of claim 5, wherein the aqueous medium is added to the oil in a volume ratio of about 1:50 to about 50:1, specifically about 1:15, more specifically about 1:1 (vol/vol).

15. (canceled)

16. The method of claim 5, wherein the oil-in-water dispersion is further diluted with an aqueous solution, such as a physiological saline solution, in a volume ratio of up to 1:6.

17. The method of claim 5, wherein, the concentration of the coniferous resin acids in the oil-in-water dispersion is in the range of about 1 ppm to about 5000 ppm, specifically about 1 ppm to about 2000 ppm, more specifically about 1 ppm to about 500 ppm, still more specifically about 10 ppm to about 100 ppm.

18. A method of using an oil-in-water dispersion of claim 1 or that prepared by a method of claim 5, wherein the dispersion is used as an antimicrobial agent in non-medical applications, such as biocides, pesticides, or foodstuffs.

19. (canceled)

20. The use method of claim 17, wherein the dispersion is applied against potato blight (Phytophthora infestans).

21. A method of using an oil-in-water dispersion of any one of claim 1 or that prepared by a method of claim 5, wherein the dispersion is used as an antimicrobial agent or an anti-inflammatory agent for treating or preventing infections and/or inflammations, such as those caused by Helicobacter pylori, in humans and animals.

22. (canceled)

23. A pharmaceutical formulation comprising an oil-in-water dispersion of claim 1 or that prepared by a method of claim 5.
